(19) United States
(12) Patent Application Publication
(10) Pub. No.: US 2024/0053312 A1
(43) Pub. Date: Feb. 15, 2024

(54) SYSTEM AND METHOD FOR DETECTING METHANE AND OTHER GASES USING A REMOTELY DEPLOYABLE, OFF-GRID SYSTEM

(71) Applicant: Champion X LLC, Sugar Land, TX (US)

(72) Inventors: Stephen Arthur Conley, Boulder, CO (US); Thomas Bleha Ryerson, Boulder, CO (US)

(21) Appl. No.: 18/269,417

(22) PCT Filed: Dec. 23, 2021
(86) PCT No.: PCT/US2021/065111

§ 371 Date: Jun. 23, 2023

Related U.S. Application Data
(63) Continuation-in-part of application No. 17/133,930, filed on Dec. 24, 2020, now abandoned.

(51) Int. Cl. G01N 33/00; G01P 5/24

(52) U.S. Cl. CPC G01N 33/0027; G01P 5/24

(57) ABSTRACT

Disclosed herein are systems and methods for detecting methane and other gases in the atmosphere. The ability to remotely deploy and autonomously detect, quantify, and report emission rates of methane and other gases to the atmosphere is an important step in the evolution of emissions calculation and reduction. This will assist energy producers, regulators, researchers, and other interested parties to better understand the emission profiles of various locations.

### FIELD OF THE INVENTION

Disclosed herein are systems and methods for detecting methane and other gases in the atmosphere. The ability to remotely deploy and autonomously detect, quantify, and report emission rates of methane and other gases to the atmosphere is an important step in the evolution of emissions calculation and reduction. This will assist energy producers, regulators, researchers, and other interested parties to better understand the emission profiles of various locations.

### BACKGROUND OF THE INVENTION

A specific need exists for autonomous and accurate detection, quantification, and automatic reporting of methane (CH₄) emissions (“leaks”) to the atmosphere. CH₄ is flammable, contributes to background ozone pollution, is a potent greenhouse gas, and is a valuable commodity. Continuous CH₄ monitoring is increasingly needed to reduce the risk of flammable leaks, identify and address sources of pollutant and greenhouse gas emissions, and to reduce saleable product losses. CH₄ emissions to the atmosphere come from a variety of natural and human sources, and national and international policies to identify and reduce these emissions is of increasing priority. Oil and gas production areas are a significant source of CH₄ to the atmosphere, but the location, timing, and magnitude of CH₄ emissions are often poorly quantified. A typical oil and gas production basin can encompass hundreds of square miles, with hundreds of thousands of potential CH₄ emission sources from the tens of thousands of well pad, gathering, and transmission facilities within a typical basin. Similarly, large concentrated animal feeding operations (CAFOs) can consist of tens of thousands of livestock, multiple sewage lagoons, large manure storage piles, and other sources of CH₄ emissions to the atmosphere. Landfills represent another significant source of CH₄ emissions whose magnitude is not well known. Finding and mitigating CH₄ emissions at their source has the potential to reduce economic losses, improve air quality, and minimize the climate impacts of the energy production, agricultural, and waste management practices needed to power, feed, and manage the output from a growing global population. Thus, inexpensive, unattended, and autonomous monitoring systems are required to provide a robust and economically feasible continuous CH₄ emissions monitoring solution suitable for extensive field deployment and operation.

Many commercially available research-grade CH₄ detectors, e.g., the Picarro model G2301 or Los Gatos model 915-0001, are optimized for ambient atmospheric measurements at ultra-trace levels well away from source regions. These detectors offer extremely high sensitivity, high selectivity for CH₄, and high instrument stability over time, but can require skilled operators, typically consume tens to hundreds of watts of AC power, and cost tens of thousands of dollars or more for each detector. These research-grade detectors are cost-prohibitive for use in a continuous emissions monitoring network set up to detect, quantify, and specifically attribute methane leaks to individual sites among thousands of facilities in an oil and gas production region, or at the thousands of CAFOs and landfills distributed throughout the U.S. A wide range of less precise, lower-cost CH₄ detectors are commercially available, for both personal exposure monitoring (e.g., the Honeywell GasAlert Extreme) and for combustible gas leak detection (e.g., the Bacharach Leakator®). These detectors are less sensitive than the research-grade detectors mentioned previously and are more prone to undesired sensitivities to gases other than CH₄ which can lead to erroneous “false positives,” especially from other combustible gases such as hydrogen, ethanol, and/or carbon monoxide. Typically, these detectors are designed for a fixed installation and require AC power or are designed to be hand-carried and require frequent battery replacement. Detector costs range from hundreds of dollars to thousands of dollars. These CH₄ detectors are not typically designed for long-term unattended use in remote locations without reliable AC power and do not provide telemetry of measured CH₄ values to a cloud-based server.

### BRIEF SUMMARY OF THE INVENTION

Disclosed herein are systems and methods for detecting and quantifying methane in an ambient atmosphere.

For example, systems for detecting and quantifying methane in an ambient atmosphere can comprise a detector comprising a housing, a methane sensor, typically a low-cost metal-oxide semiconductor sensor, a temperature sensor, a relative humidity sensor, and a data memory device operably interfaced with the methane sensor, the temperature sensor, and the relative humidity sensor; a data server; a telemetry module adapted to establish a connection between the detector and the data server and communicate sensed data to the data server; a power source operably interfaced with the methane sensor, the temperature sensor, the relative humidity sensor, the data memory device, and the telemetry module; and an energy storage device operably interfaced with the power source. The telemetry module is operably interfaced with the data server and the data memory device; the data memory device is operably interfaced with the data server for storing sensor data; and the data from the methane sensor, the temperature sensor, and the relative humidity sensor is calibrated to compensate for interference and convert methane sensor data into methane concentrations.

The systems described herein can have the detector further comprise a carbon monoxide, a hydrogen sulfide, and/or a total volatile organic compound sensor operably interfaced with the data memory device and with the power source.

The systems can also have the detector further comprise a sensor capable of sensing wind speed and wind direction operably interfaced with the data memory device and with the power source.

The systems can further have the sensor capable of sensing wind speed and wind direction comprise an ultrasonic sensor.

The systems described herein can have the telemetry module comprise a telemetry communication circuit.

The systems can also have the data server be a cloud-based data server.

The systems have the power source comprise an AC source or a renewable power source deriving energy from solar or wind.

The renewable power source can comprise a photovoltaic cell.

The systems can have the energy storage device comprise a battery.

The systems can also comprise two or more detectors.

The systems having two or more detectors can have each of the two or more detectors be located in a known position surrounding a facility or an area of interest.

The systems can also comprise three or more detectors, wherein the three or more detectors are located in a known position.

The disclosure further describes methods for detecting and quantifying methane in an ambient atmosphere, the method comprising sensing methane in the ambient atmosphere using a methane sensor; sensing temperature of the ambient atmosphere using a temperature sensor; sensing relative humidity of the ambient atmosphere using a relative humidity sensor; saving the methane sensor data, the temperature sensor data, and the relative humidity sensor data to a data memory device; transmitting the sensor data from the data memory device to a data server via cellular or wireless communication; and calibrating the methane sensor data, the temperature sensor data, and the relative humidity sensor data to compensate for interference and convert the methane sensor data into methane concentrations.

The methods described herein can further comprise sensing carbon monoxide and/or hydrogen sulfide in the ambient atmosphere using separate electrochemical-cell sensors, and can further comprise sensing total volatile organic compounds in the ambient atmosphere using a separate metal-oxide semiconductor or electrochemical cell sensor.

The methods can also further comprise sensing ambient wind speed and wind direction.

The methods can also have the chemical sensor data, temperature data, and relative humidity data be collected about once to about 50 times per second.

The ambient wind speed and wind direction data can be collected about once per second.

The methods can have one minute averages and standard deviations of wind speed and wind direction calculated and transmitted every one to 15 minutes to the data server.

The methods have the calibrated methane concentrations, the wind speed, and the wind direction variability used in an atmospheric plume dispersion model to produce methane leak rates as a function of time.

The methods described herein can have two or more sets of sensors having a known location be used, each set comprising a methane sensor, a temperature sensor, a relative humidity sensor, and optionally a carbon monoxide, hydrogen sulfide, and/or a total volatile organic compound sensor(s).

Further, the methods can have three or more sets of sensors having a known location be used.

The methods can also have one of the sets of sensors further comprise a sensor to detect wind speed and wind direction.

Other objects and features will be in part apparent and in part pointed out hereinafter.

### BRIEF DESCRIPTION OF THE SEVERAL VIEWS OF THE DRAWING

FIG. 1 shows a schematic diagram showing major components (exhaust vent, battery, solar controller, sensor and processor board, and intake fan) within the outer solar-powered detector box enclosure; the version using external AC power lacks the battery and controller. Internal and external wiring and connectors are not shown.

FIG. 2 shows a pole-mounted installation with wind sensor, detector box, and solar panel.

FIG. 3 shows a representation of the system dashboard showing an overhead view of an instrumented facility with locations of eight detector boxes (numbered circles) and facility components (squares). The footprint for each 15-minute period is automatically generated from wind direction and variability data and shown as a shaded triangle upwind of the detector box registering a leak. In this example, the system automatically and correctly identified the southwestern tank (indicated by the arrow) as the most probable leak source.

Corresponding reference characters indicate corresponding parts throughout the drawings.

### DETAILED DESCRIPTION OF THE INVENTION

Disclosed herein are systems and methods for detecting methane and other gases in the atmosphere. The ability to remotely deploy and autonomously detect, quantify, and report emissions of methane and other gases to the atmosphere is an important step in the evolution of emissions calculation and reduction. This will assist energy producers, regulators, researchers, and other interested parties to better understand the emission profiles of various locations. Unlike more traditional methods that provide an emission profile at a particular point in time and/or provide a concentration level with little or no insight into the emissions profile outside of the particular time of measurement and the actual emission rate, this system will provide a more complete emission profile by capturing emission information on a continuous basis and providing an actual estimated emission rate, including calibrations that correct for factors that can impact the emission calculation such as temperature, relative humidity, and atmospheric dispersion.

The autonomous nature of these systems and methods further allows for the continuous monitoring of facilities to occur without the need to have personnel on-site, allowing increased levels of information related to emissions without the need to increase staffing. These systems and methods will allow a user to automatically learn of a situation at a particular remote facility that is of interest and/or may require attention in near real-time rather than the more traditional approach whereby emissions may go for days, weeks, or months without being detected or addressed.

This invention relates generally to a system and method for autonomously detecting, quantifying, and automatically reporting CH₄ leaks to the atmosphere. The invention relates more specifically to a system and method that uploads data autonomously to a central server from one or more CH₄ sensor devices located on the fence line around a CH₄ source, each device consisting of a CH₄ sensor corrected for cross-sensitivities to interfering gases and ambient temperature for improved CH₄ leak detection, quantification, and source identification and reporting, said system being capable of off-grid operation by means of solar, wind, or some other renewable energy source that charges an on-board battery.

For example, systems for detecting and quantifying methane in an ambient atmosphere can comprise a detector comprising a housing, a methane sensor, a temperature sensor, a relative humidity sensor, and a data memory device operably interfaced with the methane sensor, the temperature sensor, and the relative humidity sensor; a data server; a telemetry module adapted to establish a connection between the detector and the data server and communicate sensed data to the data server; a power source operably interfaced with the methane sensor, the temperature sensor, the relative humidity sensor, the data memory device, and the telemetry module; and an energy storage device operably interfaced with the power source. The telemetry module is operably interfaced with the data server and the data memory device; the data memory device is operably interfaced with the data server for storing sensor data; and the data from the methane sensor, the temperature sensor, and the relative humidity sensor is calibrated to compensate for interference and convert methane sensor data into methane concentrations.

Modern microfabrication technology has enabled a new class of commercially available, miniaturized, and inexpensive CH₄ sensors, typically based on metal oxide semiconductor (MOS), electrochemical cell (ECC), or infrared (IR) detection of CH₄. Examples of CH₄ sensors include the Figaro TGS 26xx-series of MOS sensors, the Alphasense CH-A3 and -A4 series of ECC sensors, and the Alphasense IRM-AT series of IR detectors. These sensors are compact (˜1 to 3 cm³ in volume) consume very little power (˜tens of milliwatts) and are sufficiently inexpensive (˜$10 to $50 per unit) to enable cost-effective large-scale deployment. Drawbacks to these sensors include potential interferences from non-target gases such as carbon monoxide (CO) and volatile organic compounds (VOCs) and undesired sensor responses from changes in environmental temperature (T) and water vapor (H₂O). Without the ability to specifically measure and correct the raw sensor output for interfering gases and ambient temperature changes, these MOS, ECC, or IR sensors will suffer from false positives, i.e., spurious CH₄ detection due to temperature and humidity changes or due to elevated levels of interfering chemical species. Avoiding false positives from undesired sensitivity to chemical or environmental interferences is essential to maximize the reliability of a CH₄ monitoring system, to enhance cost-effectiveness of its incorporation into a large-scale leak detection and repair system, and to accurately monitor CH₄ leaks from thousands of potential sources in remote areas.

The temperature sensor and relative humidity sensor can be a sensor that meets the power and environmental conditions of the particular system and would be well known in the field.

The detector can also optionally comprise an intake fan and an exhaust vent that provides intake of ambient air into the housing of the detector and allows for the air to contact the one or more sensors contained in the housing and be exhausted through the exhaust vent.

The systems described herein can have the detector further comprise a carbon monoxide sensor, a hydrogen sulfide sensor, and/or a total volatile organic compound sensor operably interfaced with the data memory device and with the power source.

The systems can also have the detector further comprise a sensor capable of sensing wind speed and wind direction operably interfaced with the data memory device and with the power source.

The systems can further have the sensor capable of sensing wind speed and wind direction comprise an ultrasonic sensor.

The systems described herein can have the telemetry module comprise a telemetry communication circuit.

The systems can also have the data server be a cloud-based data server.

The systems have the power source comprise an AC power source or a renewable power source deriving energy from solar or wind.

The renewable power source can comprise a photovoltaic cell.

The systems can have the energy storage device comprise a battery.

The systems can also comprise two or more detectors.

The systems having two or more detectors can have each of the two or more detectors be located in a known position surrounding a facility or an area of interest.

The systems can also comprise three or more detectors, wherein the three or more detectors are located in a known position.

The present invention is an integrated hardware and software system that consists of one or more pole-mounted detector boxes installed at fixed locations around the perimeter of a facility to be monitored communicating with cloud-based software for data processing and information dissemination. Each detector box can be solar powered for off-grid use and has sufficient on-board battery capacity for several days of operation without charging. GPS coordinates are determined for each detector box at installation, along with coordinates for components of interest (wellheads, tanks, separators, flares, etc.) at the monitored facility. Once powered, the system continuously detects CH₄ using an inexpensive, commercially available metal-oxide semiconductor (MOS) sensor. Ancillary measurements of ambient temperature and humidity are included in each detector box, and measurements of carbon monoxide, hydrogen sulfide, and total volatile organic compounds are optionally available in each detector box. One box at each monitored facility is equipped with an ultrasonic sensor to measure horizontal wind speed and direction. Chemical sensor voltages, ambient temperature, and relative humidity data are sampled multiple times per second and wind speed and direction data are sampled once per second. 1-minute averages and the standard deviation of the wind direction are calculated, encrypted, and transmitted every 5 minutes by an embedded microprocessor equipped with either a cellular, wifi, or long-range (LoRa) radio to a cloud-based server. Software on the server applies calibrations to compensate for cross-sensitivity to temperature, humidity, and other gases to convert CH₄ sensor voltages into CH₄ mixing ratios in parts per million (ppm), which are logged and displayed along with the ancillary data as a time series on a browser-accessible dashboard. Data are encrypted and available for download in various formats by authenticated users.

The disclosure further describes methods for detecting and quantifying methane in an ambient atmosphere, the method comprising sensing methane in the ambient atmosphere using a metal-oxide semiconductor sensor; sensing temperature of the ambient atmosphere using a temperature sensor; sensing relative humidity of the ambient atmosphere using a relative humidity sensor; saving the methane sensor data, the temperature sensor data, and the relative humidity sensor data to a data memory device; transmitting the sensor data from the data memory device to a data server via cellular or wireless communication; and calibrating the methane sensor data, the temperature sensor data, and the relative humidity sensor data to compensate for interference and convert the methane sensor data into methane concentrations.

The methods described herein can further comprise sensing carbon monoxide, hydrogen sulfide, and/or total volatile organic compounds in the ambient atmosphere using separate MOS, ECC or IR sensor(s).

The methods can also further comprise sensing ambient wind speed and wind direction.

The methods can also have the methane sensor data, temperature data, and relative humidity data be collected about once to about 50 times per second; about once to about 25 times per second; about once to about 10 times per second; about once to about 5 times per second; about once to about 2 times per second.

Preferably, the ambient wind speed and wind direction data can be collected about once per second.

The methods can have one minute averages and standard deviations of wind speed and wind direction are calculated and transmitted every one to 15 minutes, or every one to 10 minutes to the data server.

The methods have the calibrated methane concentrations, the wind speed, and the wind direction variability used in an atmospheric plume dispersion model to produce methane leak rates as a function of time.

The methods described herein can have two or more sets of sensors having a known location be used, each set comprising a methane sensor, a temperature sensor, a relative humidity sensor, and optionally a carbon monoxide, a hydrogen sulfide, and/or a total volatile organic compounds sensor.

Further, the methods can have three or more sets of sensors having a known location be used.

The methods can also have one of the sets of sensors further comprise a sensor to detect wind speed and wind direction.

The methods described herein can provide continuous monitoring of the concentrations of methane and other gases of interest in a particular area of interest.

Deriving and displaying CH₄ leak rates, rather than just CH₄ concentrations, is a crucial step that greatly enhances the information provided by a continuous monitoring system, offering a more accurate picture of actual leak size by normalizing the effects of atmospheric dispersion on concentration. The system software automatically incorporates wind speed, wind direction variability, and derived atmospheric stability parameters as input to an atmospheric plume dispersion model (e.g., van Ulden, Atmospheric Environment, 1978) to calculate, log and display 15-minute-averaged CH₄ leak rates as a time series plot in the dashboard. Errors in simulating atmospheric dispersion increase during periods of light and variable winds, so CH₄ leak rates are not reported for atmospheric conditions that exceed set thresholds, e.g., wind speeds below 0.4 meter per second and 15-minute-average wind direction variability in excess of ±45°.

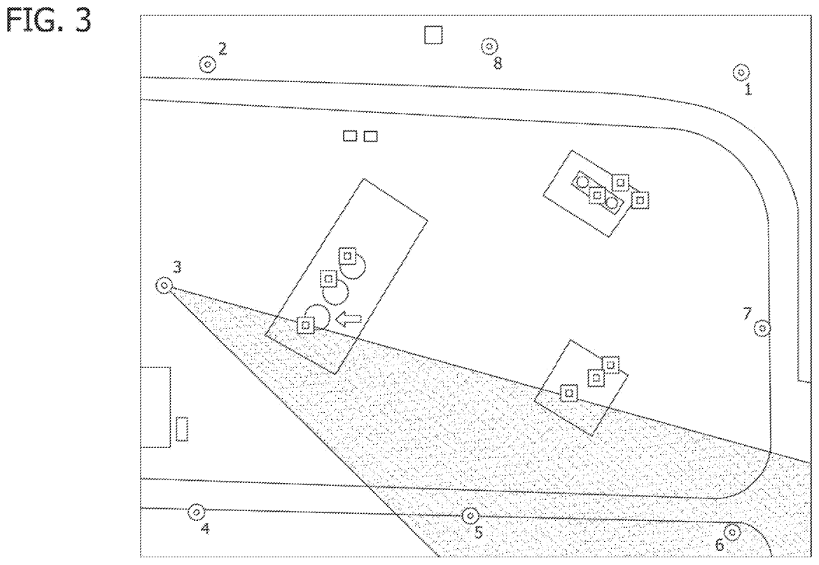

The usefulness of a targeted leak detection and repair (LDAR) program depends critically on the ability of a continuous monitoring system to reliably detect and quantify methane leak rates, identify probable sources, and alert operators to any leaks that rise above some actionable threshold for a given facility. We emphasize that methane leak rates represent the actionable information required for guiding LDAR decision making. Chemical sensors only detect methane concentrations, and variations in wind speed and atmospheric turbulence can cause methane concentrations to vary independently of the actual size of the leak rate. The present system incorporates wind speed and atmospheric turbulence into an atmospheric dispersion model to transform methane concentrations into the methane leak rates needed to guide LDAR decision making. In practice the actionable methane leak rate threshold can vary with facility size, operator requirements, applicable regulations, and other practical considerations. The present system alert threshold is fully user-configurable but by default sends automated text or email alerts to operators when a 4-hour running mean of CH₄ leak rates in excess of two standard deviations above a 30-day running mean is detected at a monitored facility. Additional calculations use average wind direction and its 15-minute variability to generate an approximate upwind source footprint and automatically identify potential source locations at the monitored facility. Automated alert information transmitted to the operator includes the facility name, location, leak rate and its uncertainty, and the most probable component(s) to which the leak is attributed as a guide for LDAR team response. Source identification accuracy depends on atmospheric transport conditions and improves over time, especially when leak detection by two or more detector boxes permits triangulation to a specific source location or facility component. For each 15-minute average leak rate calculation, the system also generates an overhead representation of facility component locations, detector box locations, and the calculated upwind footprint for the leak to provide a visual representation to guide LDAR team decision making (FIG. 3).

The present invention simultaneously measures ambient temperature (T), ambient relative humidity (RH) and optionally ambient carbon monoxide (CO), hydrogen sulfide (H₂S), and/or total volatile organic compounds (TVOCs), and corrects the raw methane sensor signal to account for these confounding factors, maximizing methane accuracy and reduce the incidence of “false positive” leak reports. For example, the temperature dependence of the methane sensor is removed by (1) multiplying the sensed temperature by a previously determined calibration coefficient, (2) subtracting the resulting value from the methane sensor raw output voltage, and (3) using the corrected output voltage to calculate the methane concentration.

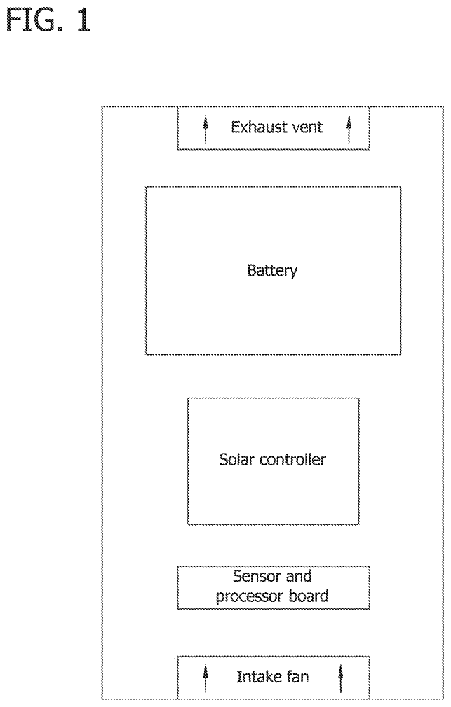

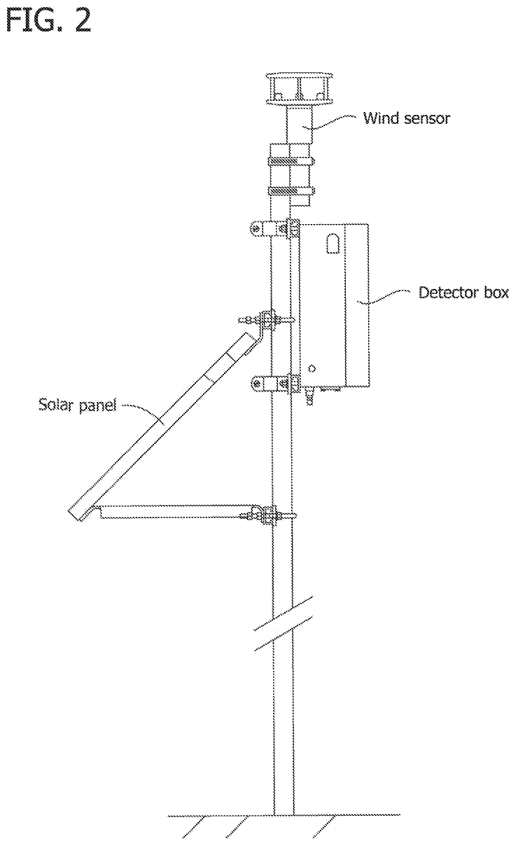

One or more methane detector boxes (FIG. 1) are installed on poles (FIG. 2) around the perimeter of a monitored facility (FIG. 3) to detect methane leaks at the fence line regardless of the prevailing wind direction. At least one box installed at a given methane source location equipped with a wind speed and direction sensor, typically, a sonic anemometer, to permit a leak to be attributed to that site, and the leak rate estimated from measured methane concentration data using mass balance calculations and a plume diversion model.

All detector boxes include an automated remote communication ability via cellular, wifi, or LoRa radio link to a central cloud-based server. Detector boxes of the present invention typically accept power from a solar, wind, or other renewable energy source that charges an on-board battery for continuous, unattended, remote, off-grid operation. Optionally, detector boxes can accept grid-tied AC or DC input power where available.

Data upload is managed to reduce transmission events and the majority of data processing takes place on the cloud-based server to decrease power consumption by the detector box. Power consumption is minimized throughout the detector box by selecting a low-power fan and microprocessor, leading to an overall continuous power draw of <5 W to maximize off-grid uptime for a given renewable power configuration.

Once data are transmitted to the cloud, the system calculates 1-minute-average chemical mixing ratios and applies an atmospheric dispersion model to derive 15-minute-average leak rates. These data are archived, displayed as time series plots on the dashboard, and made available for download. For each detector box that registers a leak, the system uses measured wind direction and its variability to calculate an upwind source area (“footprint”) for each 15-minute period and generates an overhead representation for visualization (FIG. 3).

The system further identifies facility components within the footprint of each detected leak to identify those most likely to be the source. Finally, the system alert threshold is user-configurable but by default sends automated text or email alerts when a 4-hour running mean of CH₄ leak rates in excess of 2 standard deviations above a 30-day running mean is detected at a monitored facility. Automated alert information typically includes the facility name, location, leak rate and its uncertainty, and the most probable component(s) to which the leak is attributed as a guide for LDAR team response.

The following embodiments also describe the systems and methods.

Embodiment 1. A device to selectively and accurately quantify atmospheric methane (CH₄) concentrations, comprised of one or more low-power, low-cost CH₄ sensors, a temperature sensor, a relative humidity sensor, a data logger, a telemetry capability for wireless communication off-site to a cloud-based server, a renewable power source and battery for unattended, remote, off-grid operation.

Embodiment 2. Optionally, the device of embodiment 1, further comprising one or more low-power; low-cost sensors for other gases of interest, e.g., hydrogen sulfide (H₂S), and/or potential interferences in the CH₄ measurement; e.g., carbon monoxide (CO), hydrogen (H₂), methanol (CH₃OH), ethanol (CH₃CH₂OH), acetone ((CH₃)₂(CO)), and other hydrocarbons such as ethane (C₂H₆), propane (C₃H₈), isomers of butane (C₄H₁₀), and longer-chain hydrocarbons, together summed as total volatile organic compounds (TVOCs).

Embodiment 3. The device of embodiment 1, further comprising a wind speed and direction sensor.

Embodiment 4. Cloud-based software to archive the data, process detector signals, apply calibration data to calculate chemical mixing ratios, and derive leak rates, as well as software that displays raw and processed data as time series, permits data download in various file formats, and produces geolocated results showing probable leak. locations and magnitudes, and finally, software that produces automated alerts. triggered from calculated leak rates that exceed user-selectable threshold values.

Embodiment 5. The application of a network of multiple devices and servers as described in embodiments 1, 2, 3, and 4 installed to enable fenceline monitoring of gas emissions to the atmosphere, for unattended, automated, off-grid leak detection, quantification, and automatic reporting from a multitude of remote sites.

Having described the invention in detail, it will be apparent that modifications and variations are possible without departing from the scope of the invention defined in the appended claims. Additionally, although the description above contains many specificities, these should not be construed to limit the scope of the utility of this capability but as merely providing illustrations of some of several uses.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above systems and methods without departing from the scope of the disclosure, it is intended that all matter contained in the above description and shown in the accompanying drawings shad be interpreted as illustrative and not in a limiting sense.

1. A system for detecting and quantifying methane in an ambient atmosphere, the system comprising:
a detector comprising a housing, a methane sensor, a temperature sensor, a relative humidity sensor, and a data memory device operably interfaced with the methane sensor; the temperature sensor, and the relative humidity sensor;
a data server;
a telemetry module adapted to establish a connection between the detector and the data server and communicate sensed data to the data server;
a power source operably interfaced with the methane sensor, the temperature sensor, the relative humidity sensor, the data memory device, and the telemetry module; and
an energy storage device operably interfaced with the power source;
wherein the telemetry module is operably interfaced with the data server and the data memory device; the data memory device is operably interfaced with the data server for storing sensor data; and the data from the methane sensor, the temperature sensor, and the relative humidity sensor is calibrated to compensate for interference and convert methane sensor data into methane concentrations.

2. The system of claim 1, wherein the detector further comprises a carbon monoxide sensor, a hydrogen sulfide sensor, and/or a total volatile organic compounds sensor operably interfaced with the data memory device and with the power source.

3. The system of claim 1 or 2, wherein the detector further comprises a sensor capable of sensing wind speed and wind direction operably interfaced with the data memory device and with the power source.

4. The system of claim 3, wherein the sensor capable of sensing wind speed and wind direction comprises an ultrasonic sensor.

5. The system of any one of claims 1 to 4, wherein the telemetry module comprises a telemetry communication circuit.

6. The system of any one of claims 1 to 5, wherein the data server is a cloud-based data server.

7. The system of any one of claims 1 to 6, wherein the power source comprises an AC or DC power source or a renewable power source deriving energy from solar or wind.

8. The system of claim 7, wherein the renewable power source comprises a photovoltaic cell.

9. The system of any one of claims 1 to 8, wherein the energy storage device comprises a battery.

10. The system of any one of claims 1 to 9, comprising two or more detectors.

11. The system of claim 10, wherein each of the two or more detectors is located in a known position surrounding a facility or an area of interest.

12. The system of claim 10 or 11, comprising three or more detectors.

13. A method for detecting and quantifying methane in an ambient atmosphere, the method comprising:
sensing methane in the ambient atmosphere using a MOS, ECC, or IR sensor;
sensing temperature of the ambient atmosphere using a temperature sensor;
sensing relative humidity of the ambient atmosphere using a relative humidity sensor;
saving the methane sensor data, the temperature sensor data, and the relative humidity sensor data to a data memory device;
transmitting the sensor data from the data memory device to a data server via cellular or wireless communication; and
calibrating the methane sensor data, the temperature sensor data, and the relative humidity sensor data to compensate for interference and convert the methane sensor data into methane concentrations.

14. The method of claim 13, further comprising sensing carbon monoxide, hydrogen sulfide, and/or total volatile organic compounds in the ambient atmosphere using MOS, ECC, or IR sensors.

15. The method of claim 13 or 14, further comprising sensing ambient wind speed and wind direction.

16. The method of any one of claims 13 to 15, wherein the methane sensor data, temperature data, and relative humidity data are collected about once to about 50 times per second.

17. The method of claim 15 or 16, wherein ambient wind speed and wind direction data are collected about once per second.

18. The method of any one of claims 15 to 17, wherein one minute averages and standard deviations of wind speed and wind direction are calculated and transmitted every one to 15 minutes to the data server.

19. The method of any one of claims 15 to 18, wherein the calibrated methane concentrations, the wind speed, and the wind direction variability are used in an atmospheric plume dispersion model to produce methane leak rates as a function of time.

20. The method of any one of claims 13 to 19, wherein two or more sets of sensors having a known location are used, each set comprising a metal-oxide semiconductor, a temperature sensor, a relative humidity sensor, and optionally a carbon monoxide sensor.

21. The method of claim 20, wherein three or more sets of sensors having a known location are used.

22. The method of claim 20 or 21, wherein one of the sets of sensors further comprises a sensor to detect wind speed and wind direction.
